(19) United States
(12) Patent Application Publication
(10) Pub. No.: US 2014/0336495 A1
(43) Pub. Date: Nov. 13, 2014

(54) MEDICAL IMAGING DEVICE WITH DISINFECTION UNIT

(71) Applicant: SIEMENS AKTIENGESELLSCHAFT, Munchen (DE)

(72) Inventor: GERHARD BITTNER, ERLANGEN (DE)

(73) Assignee: SIEMENS AKTIENGESELLSCHAFT, Munchen (DE)

(21) Appl. No.: 14/268,037

(22) Filed: May 2, 2014

(30) Foreign Application Priority Data
May 7, 2013 (DE) 102013208340.8

(51) Int. Cl. A61L 2/10; A61B 5/055; A61B 19/00

(52) U.S. Cl. CPC A61L 2/10; A61B 19/34; A61B 5/055
USPC 600/410; 600/407

(57) ABSTRACT

A medical imaging device with a detector unit and an acquisition unit for an examination object, wherein the acquisition unit is at least partially surrounded by the detector unit, and at least one disinfection unit for the disinfection of surfaces of the medical imaging device, wherein the disinfection unit has at least one UV-unit and the acquisition unit is arranged at least partially within the radiation range of the UV-unit is provided.

### CROSS REFERENCE TO RELATED APPLICATIONS

This application claims priority to DE Application No. 102013208340.8, having a filing date of May 7, 2013, the entire contents of which are hereby incorporated by reference.

### FIELD OF TECHNOLOGY

The following relates to a medical imaging device with a disinfection unit and a disinfection unit.

### BACKGROUND

In everyday clinical operation, the surfaces of medical imaging devices can become contaminated with living microorganisms such as for example bacteria, spores, fungi and/or viruses, by, inter alia, contact with a patient. This increases the danger of the infection of other patients.

In order to reduce the spread of such microorganisms, the surfaces of the medical imaging devices are for example covered with cloths.

The embodiments effectively clean the surfaces of medical imaging devices after examinations performed on patients and thereby help facilitate an unhindered progress of clinical operation.

This problem is solved by a medical imaging device and a disinfection unit. Exemplary embodiments of the invention are specified in each case.

### SUMMARY

The invention uses an ultraviolet unit (UV-unit) of a disinfection unit, in order effectively to eliminate microorganisms by means of irradiation with the UV-unit.

A medical imaging device is here provided with a detector unit and an acquisition unit for an examination object, wherein the acquisition unit is at least partially surrounded by the detector unit and at least one disinfection unit for disinfection of surfaces of the medical imaging device, wherein the disinfection unit has at least one UV-unit and the acquisition unit is arranged at least partially within a radiation range of the UV-unit. The radiation range is in this case that area captured by the radiation of the UV-unit, that is an object within this radiation range is also reached by the UV rays.

By means of the inventive arrangement of the disinfection unit areas of the medical devices, such as for example accommodating areas for patients, such as a tunnel of a magnetic resonance device and/or parts of the devices, such as for example body coils in the case of magnetic resonance devices and/or device surfaces, such as for example patient couches, can be captured by radiation from the UV-unit and thus effectively cleaned. The transmission of pathogens across the surfaces of the medical device is thereby limited.

In one exemplary embodiment the UV-unit of the medical imaging device comprises an arrangement of UV-LEDs. These can be operated within a wavelength range between 245 nm, 265 nm, and 253.7 nm. This corresponds to the wavelength of the primary radiation of a low-pressure mercury vapor discharge. Thanks to the small dimensions of the UV-LEDs, these can be arranged in virtually any position and in any quantity.

In one exemplary embodiment the disinfection unit can be optimized to a surface to be disinfected, so that the desired surface area can also be irradiated with UV radiation.

In a further embodiment the optimization of the disinfection unit comprises an optimization of a distance between the disinfection unit and the surface to be disinfected. By means of a greater distance a larger surface area can be irradiated, while by means of a smaller distance on the other hand, the radiation intensity per unit of surface can be increased. Intensively soiled surfaces can for example be irradiated from a lesser distance, less heavily soiled surfaces correspondingly from a greater distance. The optimization of the distance is thus equivalent to the possibility of setting a distance which is favorable in relation to the surface to be disinfected and/or further outline conditions.

In one inventive embodiment the optimization of the disinfection unit is based on an optimization of a deployment time of the disinfection unit to the surface to be disinfected. The deployment time can for example be adjusted to the degree of soiling and/or a clinical schedule. Intensively soiled surfaces can for example be irradiated for longer, and less heavily soiled surfaces for a correspondingly shorter period. On the other hand, during breaks in the operation of the medical device, the disinfection unit can, for example, be constantly switched on. The optimization of the deployment time thus equates to the possibility of setting an irradiation period which is favorable in relation to be disinfected surface and/or further outline conditions.

In one exemplary embodiment, the disinfection unit is connected to the detector unit, which means in particular that disinfection unit and detector unit are embodied in one piece. The disinfection can thereby be combined with the clinical deployment of the detector unit in a particularly time-saving manner.

In one exemplary embodiment the disinfection unit is arranged on a bracket outside the acquisition unit and/or the detector unit. Brackets of this kind may, but need not necessarily take, inter alia, the form of tubes or struts, which enable the affixing of the disinfection unit to the acquisition unit and/or the detector unit. Additionally, however, brackets of this kind also enable the disinfection unit to be attached to, and detached from the acquisition unit and/or the detector unit.

In a further embodiment the bracket is embodied in the detector unit in retractable form, that is to say that the bracket can for example be slid into an accommodating area of the detector unit specifically intended for the bracket. This enables an additional, flexible integration of the disinfection unit. The bracket can here for example be mechanically retractable or embodied in movable form by means of a motor. In this way it is possible to move the disinfection unit in a flexible and precise manner when in use, or as the case may be to retract it again when out of use. Different surfaces of the medical imaging device can also for example be irradiated according to a prescribed program sequence. Surfaces outside of the accommodating area can also be disinfected thereby. In one inventive embodiment a patient couch of the medical imaging device is arranged at least partially within the radiation range, in order also to be able efficiently to disinfect the direct placement location of a patient.

In one exemplary embodiment the disinfection unit comprises a motion sensor, which can in particular detect the movements of people. In this way it can be ensured that the irradiation with the UV-unit is suspended when the motion sensor is triggered, for example by the movement of a person, so that the latter does not come to harm as a result of the UV radiation. The safety of the disinfection unit is thereby enhanced.

In exemplary embodiments the detector unit of the medical imaging device comprises a disinfection mode. By means of an operating mode specially oriented towards disinfection it can for example be ensured that during the disinfection no medical imaging is possible or, as the case may be, no disinfection can be performed during the medical imaging. This serves to improve safety for the patient and the medical personnel alike. If the medical device takes the form of a magnetic resonance device (also known as an MR device), the coupling of high frequency and pulsating magnetic fields can be avoided, if the UV-LEDs are brought into the accommodating area only for disinfection with a deactivated MR system.

In one exemplary embodiment the disinfection unit is embodied such that it can be switched on and off. Thereby too, it is possible to differentiate between two operating modes of the disinfection unit. In addition the disinfection unit can be switched off if required and the disinfection mode interrupted, if, for example, other safety methods are not available.

In one exemplary embodiment the medical imaging device comprises a magnetic resonance device. In this way a disinfection of magnetic resonance devices with all their special embodiments can be achieved. Magnetic resonance devices with high field strengths, such as for example 7 Tesla, can thus be disinfected without exposing humans to the powerful magnetic field.

In a further embodiment the disinfection unit comprises lines, which for a supply voltage to the disinfection unit are geared towards the operation of the magnetic resonance device. The supplying of power, in particular to UV-LEDs which are located in a strong magnetic field of a magnetic resonance devices, cannot take place using standard power lines, since as a result of the pulsating magnetic fields and high-frequency fields voltages are induced which cause the UV-LEDs to emit radiation or can even destroy them. The power supply thus takes place, for example, with filters made up of chokes and condensers, suitable cable routing and/or by means of twisting of the lines.

Within the framework of the embodiments a disinfection unit is also provided, which is designed to disinfect an acquisition unit of a medical imaging device. The disinfection unit here comprises an UV-unit and a bracket, on which the UV-unit is arranged, wherein the disinfection unit can be introduced into the acquisition unit by means of the bracket, and wherein the UV-unit comprises at least one UV-LED, which can be operated within a wavelength range between 245 nm and 265 nm. By means of a disinfection unit of this kind, not a priori connected to a medical device, multiple medical devices of the same embodiment for example can be disinfected in a sequence, in that the disinfection unit is used as a mobile unit. This flexible utilization is in addition more cost-effective.

In one exemplary embodiment of the disinfection unit the at least one UV-LED can be operated within a wavelength range between 250 nm, 255 nm, and 253.7 nm. This corresponds to the wavelength of the primary radiation of a low-pressure mercury vapor discharge.

### BRIEF DESCRIPTION

Some of the embodiments will be described in detail, with reference to the following figures, wherein like designations denote like members wherein.

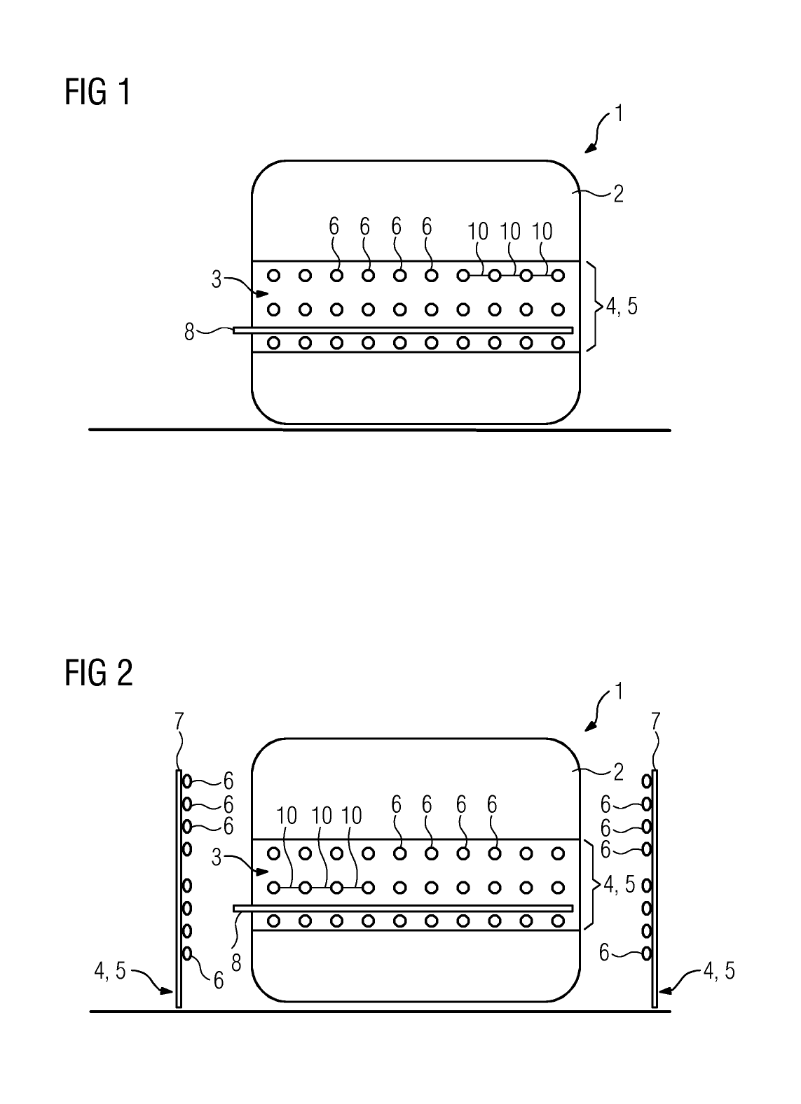

FIG. 1 depicts a medical imaging device with disinfection unit;

FIG. 2 depicts an alternative embodiment of a medical imaging device with disinfection unit; and

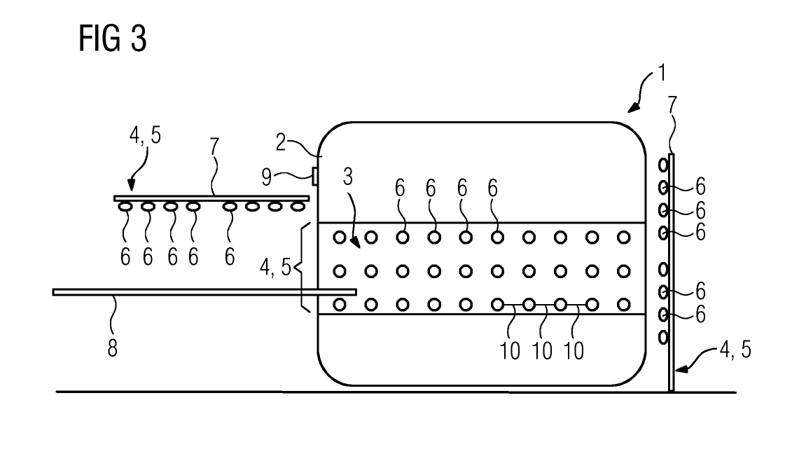

FIG. 3 depicts a further embodiment of a medical imaging device with disinfection unit.

### DETAILED DESCRIPTION

FIG. 1 shows a medical imaging device 1, here a magnetic resonance device, with a detector unit 2 and an acquisition unit 3 for an examination object. Instead of the magnetic resonance device, the use of a combined magnetic resonance positron emission tomography device, a computed tomography device or another device which can be used for other imaging procedures is also possible. Depending on the medical imaging device 1 employed, the detector unit 2 can for example also comprise an excitation unit, a radiation generating unit and/or a magnet unit.

The acquisition unit 3 may be partially surrounded by the detector unit 2 and a disinfection unit 4 for the disinfection of surfaces of the medical imaging device 1. The disinfection unit 4 has a UV-unit 5 and the acquisition unit 3 is arranged at least partially within a radiation range of the UV-unit 5.

The UV-unit 5 comprises an arrangement of UV-LEDs 6. These can be operated within a wavelength range between 245 nm, 265 nm, and 253.7 nm. This corresponds to the wavelength of the primary radiation of a low-pressure mercury vapor discharge. The disinfection unit 4 can be optimized to a surface to be disinfected, so that the desired surface area can be irradiated.

The disinfection unit 4 is connected to the detector unit 2, as a result of which the disinfection can be combined with the clinical deployment of detector unit 2 in a particularly time-saving manner. The radiation range also includes a patient couch 8 of the medical imaging device 1.

The disinfection unit 4 shown comprises lines 10, which for a supply voltage to the disinfection unit 4 are geared towards the operation of the magnetic resonance device, so that no voltages are induced which can cause the UV-LEDs 6 to emit radiation or even destroy them. The power supply thus takes place, for example, with filters made up of chokes and condensers, suitable cable routing and/or by means of twisting of the lines 10.

The disinfection unit 4 shown here is part of the medical imaging device 1, the disinfection unit 4 can however also be embodied as a separate unit, which is designed to disinfect an acquisition unit 3 of a medical imaging devices 1, with a UV-unit 5 and a bracket 7, on which the UV-unit 5 is arranged, wherein by means of the bracket 7 the disinfection unit 4 can be introduced into the acquisition unit 3.

FIG. 2 and FIG. 3 show alternative exemplary embodiments of an inventive medical imaging device 1 with disinfection unit 4 to FIG. 1. Components, features and functions which remain essentially identical are in all cases numbered with the same reference characters. The descriptions which follow are essentially limited to the differences from the exemplary embodiment in FIG. 1, wherein as regards components, features and functions which remain identical, attention is drawn to the description of the exemplary embodiment in FIG. 1.

FIG. 2 shows an alternative embodiment of a medical imaging device 1 with disinfection unit 4. The disinfection unit 4 is here arranged on two brackets 7 outside the acquisition unit 3 and the detector unit 2. Brackets 7 of this kind can, for example, be tubes or struts, which enable the affixing of the disinfection unit 4 to the acquisition unit 3 and/or the detector unit 2. The bracket 7 can however also be embodied to be able to retract into the detector unit 2, that is to say that the bracket 7 can for example be slid into an accommodating area of the detector unit 2 specifically for the bracket 7.

FIG. 3 shows a further embodiment of a medical imaging device 1 with disinfection unit 4. A patient couch 8 of the medical imaging device 1 is here arranged at least partially within the radiation range, so that the patient couch 8 is also reached by the UV-rays.

It is further possible that the distance between the disinfection unit 4 and the surface to be disinfected and/or the deployment time of the disinfection unit 4 can be optimized to the surface to be disinfected. The optimization of the distance equates to the possibility of setting a distance which is favorable for the surface to be disinfected and/or further outline conditions, while the optimization of the deployment time equates to the possibility a setting an irradiation time which is favorable for the surface to be disinfected and/or further outline conditions.

The medical imaging device 1 further comprises a motion sensor 9, which can in particular detect the movements of people. In this way it can be ensured that the irradiation with the UV-unit 5 is suspended when the motion sensor 9 is triggered, for example by the movement of a person, so that the latter does not come to harm as a result of the UV radiation.

The detector unit 2 of the medical imaging device 1 comprises a disinfection mode which can be activated and deactivated, with the aid of which it can be ensured that during the disinfection no medical imaging is possible, or, as the case may be, during the medical imaging no disinfection can be performed.

To summarize, embodiments relate to a medical imaging device with a detector unit and an acquisition unit for an examination object, wherein the acquisition unit may be surrounded at least partially by the detector unit, and at least one disinfection unit for disinfection of surfaces of the medical imaging device, wherein the disinfection unit has at least one UV-unit and the acquisition unit is arranged at least partially within the radiation range of the UV-unit.

1. A medical imaging device with a detector unit and an acquisition unit for an examination object, wherein the acquisition unit is at least partially surrounded by the detector unit, and at least one disinfection unit for the disinfection of surfaces of the medical imaging device, wherein the disinfection unit has at least one UV-unit and the acquisition unit is arranged at least partially within the radiation range of the UV-unit.

2. The medical imaging device as claimed in claim 1, wherein the UV-unit comprises an arrangement of UV-LEDs.

3. The medical imaging device as claimed in claim 1, wherein the disinfection unit is optimized to a surface to be disinfected.

4. The medical imaging device as claimed in claim 1, wherein the optimization of the disinfection unit comprises an optimization of a distance between the disinfection unit and the surface to be disinfected.

5. The medical imaging device as claimed in claim 1, wherein the optimization of the disinfection unit comprises an optimization of a deployment time of the disinfection unit to the surface to be disinfected.

6. The medical imaging device as claimed in claim 1, wherein the disinfection unit is connected to the detector unit.

7. The medical imaging device as claimed in claim 1, wherein the disinfection unit is arranged on a bracket outside the acquisition unit and/or the detector unit.

8. The medical imaging device as claimed in claim 7, wherein the bracket is embodied to be retractable into the detector unit.

9. The medical imaging device as claimed in claim 1, wherein a patient couch of the medical imaging device is arranged at least partially within the radiation range.

10. The medical imaging device as claimed in claim 1, wherein the disinfection unit comprises a motion sensor.

11. The medical imaging device as claimed in claim 1, wherein the detector unit of the medical imaging devices comprises a disinfection mode.

12. The medical imaging device as claimed in claim 1, wherein the disinfection unit is embodied such that it is switched on and off.

13. The medical imaging device as claimed in claim 1, wherein the medical imaging device comprises a magnetic resonance device.

14. The medical imaging device as claimed in claim 13, wherein the disinfection unit comprises lines, which for a power supply to the disinfection unit are geared towards the operation of the magnetic resonance device.

15. A disinfection unit, which is designed to disinfect an acquisition unit of a medical imaging device, with a UV-unit and a bracket, on which the UV-unit is arranged, wherein by means of the bracket the disinfection unit is slid into the acquisition unit and wherein the UV-unit comprises at least one UV-LED, which is operated within a wavelength range between 245 nm and 265 nm.

16. The disinfection unit as claimed in claim 15, wherein at least one UV-LED is operated within a wavelength range between 250 nm, 255 nm, and 253.7 nm.
